(19) United States
(12) Patent Application Publication
(10) Pub. No.: US 2011/0218615 A1
(43) Pub. Date: Sep. 8, 2011

(54) Stent With Multi-Crown Constraint and Method for Ending Helical Wound Stents

(75) Inventor: Erik Griswold, Penngrove, CA (US)

(73) Assignee: Medtronic Vascular, Inc., Santa Rosa, CA (US)

(21) Appl. No.: 12/716,018

(22) Filed: Mar. 2, 2010

(51) Int. Cl. A61F 2/82; B23P 11/00

(52) U.S. Cl. 623/1.15; 29/428

(57) ABSTRACT

A stent includes a wave form having a plurality of struts and a plurality of crowns with each crown connecting two adjacent struts. The wave form is wrapped around a longitudinal axis to define a plurality of turns. The stent includes a first connection that connects an end of the wave form to an adjacent crown in a first turn that defines an end of the stent, and a second connection that connects a first crown of the wave form to an adjacent crown in a second turn.

### BACKGROUND OF THE INVENTION

1. Field of the Invention

The present invention is generally related to a stent and a method for manufacturing a stent. More particularly, the present invention is related to a stent with multi-crown constraint, particularly in an end portion of the stent, and a method for ending helical wound stents.

2. BACKGROUND OF THE INVENTION

A stent is typically a hollow, generally cylindrical device that is deployed in a body lumen from a radially contracted configuration into a radially expanded configuration, which allows it to contact and support a vessel wall. A plastically deformable stent can be implanted during an angioplasty procedure by using a delivery system that includes a balloon catheter bearing a compressed or “crimped” stent, which has been loaded onto the balloon. The stent radially expands as the balloon is inflated, forcing the stent into contact with the body lumen, thereby forming a support for the vessel wall. Deployment is effected after the stent has been introduced percutaneously, transported transluminally, and positioned at a desired location by means of the balloon catheter.

Stents may be formed from wire(s), may be cut from a tube, or may be cut from a sheet of material and then rolled into a tube-like structure. While some stents may include a plurality of connected rings that are substantially parallel to each other and are oriented substantially perpendicular to a longitudinal axis of the stent, others may include a helical coil that is wrapped around the longitudinal axis at a non-perpendicular angle. Helical stents tend to have ends that are not perpendicular to the longitudinal axis due to the pitch of the helix. To square off the ends of a helical stent, the last turn at either end may include a waveform that includes waves of varying amplitudes. However, by varying the amplitudes of the waves, the stent may exhibit non-uniform behavior as the stent is crimped onto a balloon and/or expanded at the deployment site.

### SUMMARY OF THE INVENTION

It is desirable to provide a stent that is configured to deploy substantially symmetrically along the length of the stent and to provide suitable scaffolding of tissue at the ends of the stent when deployed in a vessel. It is also desirable to provide a method of manufacturing such a stent.

It is an aspect of the present invention to provide a stent that includes a wave form having a plurality of struts and a plurality of crowns with each crown connecting two adjacent struts. The wave form is wrapped around a longitudinal axis to define a plurality of turns. The stent includes a first connection that connects an end of the wave form to an adjacent crown in a first turn that defines an end of the stent, and a second connection that connects a first crown of the wave form to an adjacent crown in a second turn.

It is an aspect of the present invention to provide a method of manufacturing a stent. The method includes forming a wave form having a plurality of struts and a plurality of crowns. Each crown connects two adjacent struts. The method includes wrapping the wave form about a longitudinal axis to define a plurality of turns so that an end of the wave form is positioned adjacent a crown of a first turn that defines an end of the stent, connecting the end of the wave form to the adjacent crown of the first turn, and connecting a first crown of the wave form to an adjacent crown of a second turn.

### BRIEF DESCRIPTION OF THE DRAWINGS

Embodiments of the invention will now be described, by way of example only, with reference to the accompanying schematic drawings in which corresponding reference symbols indicate corresponding parts, and in which:

FIG. 1 schematically depicts a wave form of a stent before the wave form is wrapped about a longitudinal axis to form the stent according to an embodiment of the present invention;

FIG. 2 schematically depicts a stent comprising the wave form of FIG. 1 after the wave form has been wrapped about the longitudinal axis, with the stent in an unrolled configuration;

FIG. 3 schematically depicts a stent comprising a wave form that has been wrapped about the longitudinal axis according to an embodiment of the present invention, with the stent in an unrolled configuration; and

FIG. 4 is a more detailed view of an end portion of the stent of FIG. 3.

### DETAILED DESCRIPTION OF EMBODIMENTS OF THE INVENTION

The following detailed description is merely exemplary in nature and is not intended to limit the invention or the application and use of the invention. Furthermore, there is no intention to be bound by any expressed or implied theory presented in the preceding technical field, background, brief summary or the following detailed description.

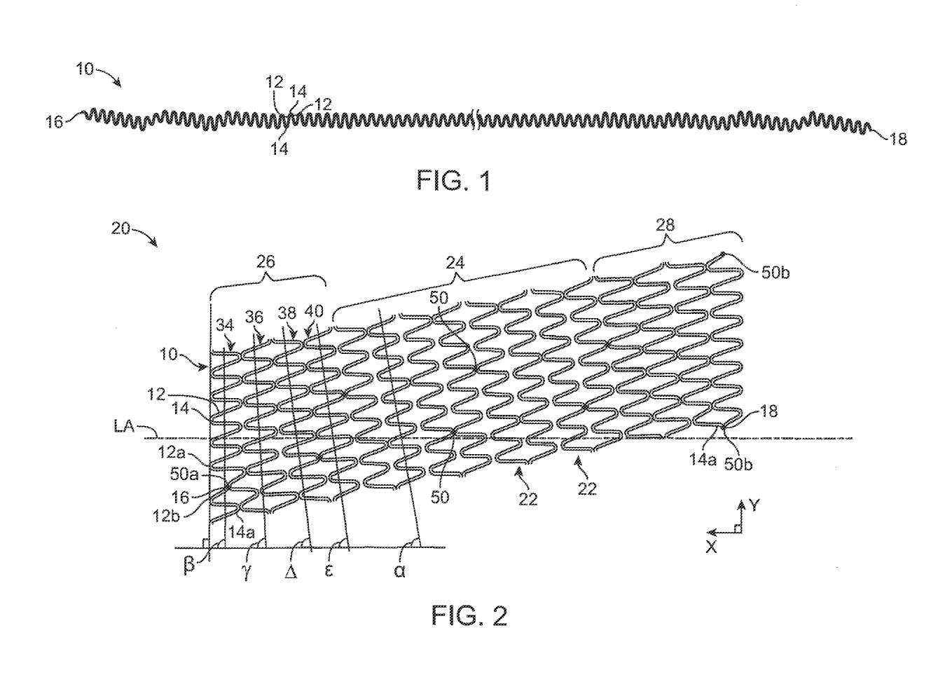

FIG. 1 illustrates a wave form 10 includes a plurality of struts 12 and a plurality of crowns 14. Each crown 14 is a curved portion or turn within the wave form 10 that connects adjacent struts 12 to define the continuous wave form 10. As shown in FIG. 1, the struts 12 are substantially straight portions of the wave form 10. In other embodiments of the invention, the struts 12 may be slightly bent or have other shapes, such as a sinusoidal wave, for example. In an embodiment, the wave form 10 may be formed from a single wire having a first end and a second end, which become a first end 16 a second end 18 of the wave form 10, respectively, as illustrated in FIG. 1.

FIG. 2 illustrates a stent 20 that may be formed from the wave form 10 of FIG. 1. Although the stent 20 is generally cylindrical in shape and has a longitudinal axis LA extending through the center of the stent 20, as known in the art, FIG. 2 illustrates the stent 20 in an “unrolled” state, which may be created when the stent 20 is slit from one end to the other end. The stent 20 includes a plurality of turns 22 that are created when the wave form 10 is wrapped around the longitudinal axis LA during manufacturing of the stent 20. A mandrel or rod that is aligned with the longitudinal axis LA may be used to support the wave form 10 as the wave form 10 is wrapped around the longitudinal axis LA. The stent 10 generally includes a central portion 24 and two end portions, a first end portion 26 and a second end portion 28, that are located on opposite sides of the central portion 24. In an embodiment, the first end portion 26 and the second end portion 28 may be mirror images of each other.

As illustrated in FIG. 2, the wave form 10 is wrapped around the longitudinal axis LA a different pitches so that the wave form 10 generally defines a helical coil in the central portion 24 having a first helical angle, or first pitch angle α, and also defines ends that are substantially square or perpendicular with the longitudinal axis LA. Specifically, crowns 14 that define the ends of the stent 20 lie in a plane that is substantially perpendicular to the longitudinal axis LA, as illustrated at the end portion 26 in FIG. 2. The first end portion 26, include a first turn 34 that is wrapped about the longitudinal axis LA at an angle β of about 90° so that the stent 20 has an end that is substantially orthogonal or perpendicular to the longitudinal axis LA.

The number of turns 22 about the longitudinal axis LA and the first helical angle a may be determined by the particular specifications of the stent 20, such as the desired unexpanded and expanded diameters and the length of the stent, as well as the size (e.g., diameter) and particular material of the wire or strip of material that may be used to create the wave form 10. The illustrated embodiment is not intended to be limiting in any way.

The first end portion 26 also includes a second turn 36 that is a continuation of the wave form 10 from the first turn 34. The second turn 36 is wrapped about the longitudinal axis LA at a second pitch angle γ that is less than 90° but greater than the first pitch angle α. Additional turns may be part of the first end portion 26, such as a third turn 38, and a fourth turn 40, and the additional turns may be configured to provide a more gradual transition between the first turn 34 that is wrapped about the longitudinal axis LA at about 90° and the first pitch angle α of the central portion 24. In the illustrated embodiment, the third turn 38 is wrapped about the longitudinal axis LA at a third pitch angle Δ, which is greater than the first pitch angle α but less than the second pitch angle γ, and the fourth turn 40 is wrapped about the longitudinal axis LA at a fourth pitch angle ε, which is greater than the first pitch angle α but less than the third pitch angle γ.

As illustrated, each of the turns 34, 36, 38, 40 of the first end portion 26 include struts 12 having different lengths, and some of the struts 12 have a length that is longer, labeled 12a in FIG. 2, than the average length of all of the struts 12 of the stent 20. It is desirable to have the length of the longest strut 12a of any given turn 34, 36, 38, 40 to be as short as possible, yet provide the desired transition in pitch angle. The presence of the longer struts 12a in the first end portion 26 allow for the transition from the orthogonal end to the helical central portion 24, but may cause the stent 20 to expand unevenly, as compared to central portion 24, when an internal pressure is applied to the stent 20. In an embodiment, some of the struts located in the first end portion 26 may have lengths that are shorter, labeled 12b in FIG. 2, than an average length of all of the struts 12 of the stent 20.

The stent 20 also includes a plurality of connections 50 that are configured to connect selected crowns 14 of adjacent turns 22. As illustrated in FIG. 2, a connection 50a is used to connect the end 16 of the wave form 10 to an adjacent crown 14a, and a connection 50b is used to connect the other end 18 of the wave form 20 to an adjacent crown 14a. The adjacent crowns 14a that are connected to the ends 16, 18 of the stent 20 of the embodiment of FIG. 2 do not define part of the ends of the stent 20, i.e., the crowns 14a do not lie in a plane that passes through the end of the stent 20, as illustrated in FIG. 2.

The connections 50 may be created by fusing the selected crowns 14 together. Similarly, the connections 50a, 50b may be created by fusing each end 16, 18 to its adjacent crown 14a, respectively. As used herein, “fusing” is defined as heating the target portions of the stent 20, e.g., the selected crowns 14, the adjacent crowns 14a, or the ends 16, 18, to be fused together, without adding any additional material, to a level where the material in the target portions flow together, intermix with one another, and form a fusion when the materials cool down to, for example, room temperature. A suitable laser may be used to create the fusion.

In an embodiment, the connections 50 may be created by welding or soldering the selected crowns 14 together. Similarly, the connections 50a, 50b may be created by welding or soldering each end 16, 18 to its adjacent crown 14a, respectively. As used herein, “welding” and “soldering” are defined as heating an additional material that is separate from the portions of the stent 20, e.g., the selected crowns 14, the adjacent crowns 14a, or the ends 16, 18, to be welded together and applying the heated additional material to the selected portions of the stent 20, so that when the additional material cools, the selected portions of the stent 20 are secured to each other.

In an embodiment, the connections 50, 50a, 50b may be created by fusing, welding, or soldering an additional piece of material (not shown) that extends between selected portions of the stent 20 to be connected. The additional piece of material may resemble a strut or a portion of a strut, and may be sized to provide spacing between the selected crowns of two adjacent turns, if desired. The illustrated embodiments are not intended to be limiting in any way.

The size of the connections 50, 50a, 50b may also be varied according to the desired flexibility and rate of expansion for a given area of the stent 20. In general, the larger the connection, i.e. the larger the fusion or weld, the greater the stiffness, and the slower the rate of expansion of the stent in the area of the larger connections.

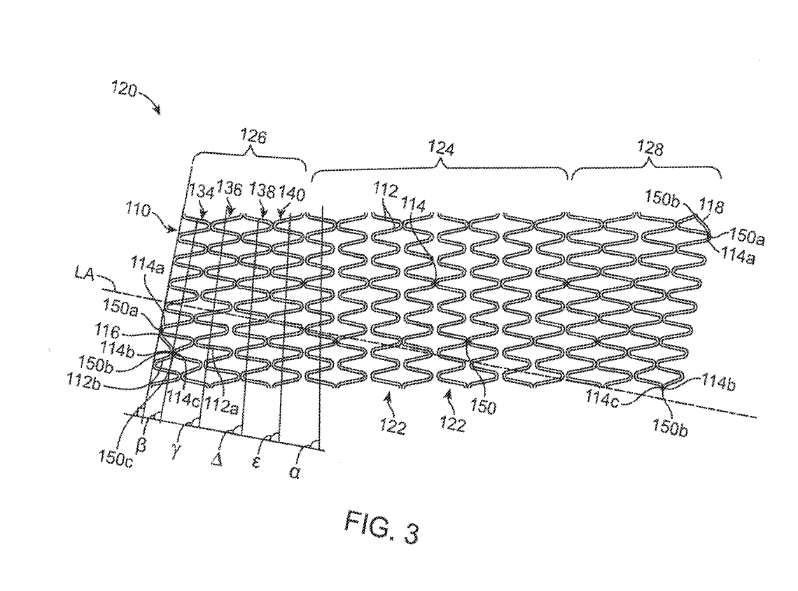

FIG. 3 illustrates a stent 120 according to an embodiment of the present invention in an “unrolled” state. The stent 120 includes a continuous wave form 110 that is similar to the wave form 10 of FIG. 1, with the exception that an additional crown and an additional strut are provided at each end of the wave form 110, as will be discussed in further detail below. The stent 120 includes a plurality of turns 122 that are created when the wave form 110 is wrapped around the longitudinal axis LA during manufacturing of the stent 120. The stent 120 generally includes a central portion 124 and two end portions, a first end portion 126 and a second end portion 128, that are located on opposite sides of the central portion 124.

As illustrated in FIG. 3, similar to the stent 20 of FIG. 2, in order to manufacture the stent 120 of FIG. 3, the wave form 110 is wrapped around the longitudinal axis LA at different pitches so that the wave form 110 generally defines a helical coil in the central portion 124 having the first helical angle, or first pitch angle α, and also defines ends that are substantially square or perpendicular with the longitudinal axis LA. As illustrated, the first end portion 126, include a first turn 134 that is wrapped about the longitudinal axis LA at an angle β of about 90° so that the stent 120 has an end that is substantially square or perpendicular to the longitudinal axis LA.

The number of turns 122 about the longitudinal axis LA and the first helical angle a may be determined by the particular specifications of the stent 120, such as the desired unexpanded and expanded diameters and the length of the stent, as well as the size (e.g., diameter) and particular material of the wire or strip of material. The illustrated embodiment is not intended to be limiting in any way.

The first end portion 126 also includes a second turn 136 that is a continuation of the wave form 110 from the first turn 134. The second turn 136 is wrapped about the longitudinal axis LA at the second pitch angle y that is less than 90° but greater than the first pitch angle α. Additional turns may be part of the first end portion 126, such as a third turn 138, and a fourth turn 140, and may be configured to provide a more gradual transition between the first turn 134 that is wrapped about the longitudinal axis LA at about 90° and the first pitch angle α of the central portion 124. In the illustrated embodiment, the third turn 138 is wrapped about the longitudinal axis LA at the third pitch angle Δ, which is greater than the first pitch angle α but less than the second pitch angle γ, and the fourth turn 140 is wrapped about the longitudinal axis LA at the fourth pitch angle ε, which is greater than the first pitch angle α but less than the third pitch angle γ.

As illustrated, each of the turns 134, 136, 138, 140 of the first end portion 126 include struts 112 having different lengths, and some of the struts 112 have a length that is longer, labeled 112a in FIG. 3, than the average length of all of the struts 112 of the stent 120. It is desirable to have the length of the longest strut 112a of any given turn 134, 136, 138, 140 to be as short as possible, yet provide the desired transition in pitch angle. The presence of the longer struts 112a in the first end portion 126 allow for the transition from the orthogonal end to the helical central portion 124, but may cause the stent 120 to expand unevenly, as compared to central portion 124, when an internal pressure is applied to the stent 120. In an embodiment, some of the struts 112 located in the first end portion 126 may have lengths that are shorter, labeled 112b in FIG. 3, than an average length of all of the struts 112 of the stent 120. Of course, any number of transition turns may be in each end portion 126, 128 to transition the helix of the central portion 124 to an orthogonal end. In an embodiment, the central portion of stent consists of a series of transitions so that the entire stent is made up of transitions and each turn includes struts of different lengths, and no two adjacent turns have the same pitch angle. The illustrated embodiments are not intended to be limiting in any way.

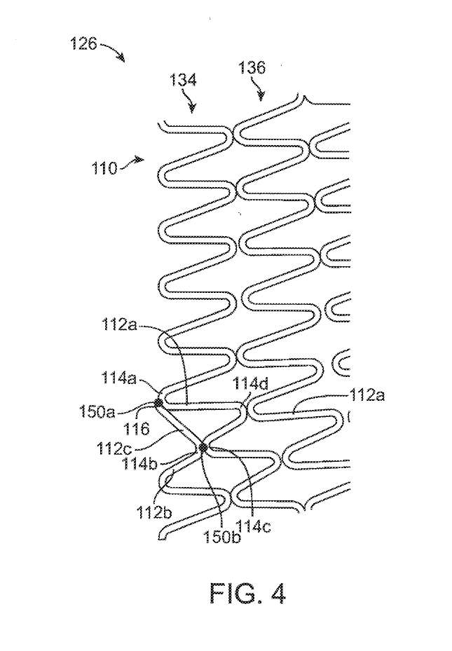

The stent 120 also includes a plurality of connections 150 that are configured to connect selected crowns 114 of adjacent turns 122. As illustrated in FIG. 3, a connection 150a is used to connect an end 116 of the wave form 110 to an adjacent crown 114a, and another connection 150a is used to connect the other end 118 of the wave form 110 to an adjacent crown 114a. As illustrated, the crowns 114a that are connected to the ends 116, 118 of the wave form 110 are located at the ends of the stent 120, i.e., the crowns 114a lie in a plane that is desirably substantially perpendicular to the longitudinal axis LA and passes through the end of the stent 120. As illustrated in FIG. 4, the crown 114a that is connected to the end 116 of the wave form 110 is also connected to one of the struts 112a that is longer than the average length of all of the struts 112 of the wave form 110.

In addition, a first crown 114b of the first turn 134 is connected to an adjacent crown 114c in the second turn 136 with a connection 150b, as illustrated in FIGS. 3 and 4, and a similar connection 150b is used at the second end portion 128, as illustrated in FIG. 3. As illustrated in FIG. 4, an additional crown 114d is located along the wave form 110 in between the crown 114a that is connected to the end 116 of the wave form 110 and the crown 114c that is connected to the first crown 114b of the wave form 110.

By comparing the end portions 26, 28 of the stent 20 of FIG. 2 with the end portions 126, 128 of the stent 120 of FIG. 3, it can be seen that an extra crown 114b, as well as an extra strut 112c, is provided at each end of the wave form 110 that is used to create the stent 120 illustrated in FIG. 3. This allows for multi-crown constraint at the end of the stent 120, which has been found to improve the uniformity of the diameter of the stent 120 along the length of the stent 120 as the stent 120 is expanded at the target deployment site, as compared to the stent 20 of FIG. 2.

The connections 150 may be created by fusing the selected crowns 114 together. Similarly, the connections 150a may be created by fusing each end 116, 118 to its adjacent crown 114a, and the connections 150b may be created by fusing the first crown 114b of the first turn 134 to the adjacent crown 114c in the second turn 136. As used herein, “fusing” is defined as heating the target portions of the stent 120, e.g., the selected crowns 114, the adjacent crowns 114a, the crowns 114, 114c, or the ends 116, 118, to be fused together, without adding any additional material, to a level where the material in the target portions flow together, intermix with one another, and form a fusion when the materials cool down to, for example, room temperature. A suitable laser may be used to create the fusion.

In an embodiment, the connections 150 may be created by welding or soldering the selected crowns 114 together. Similarly, the connections 150a may be created by welding or soldering each end 116, 118 to its adjacent crown 114a, and the connections 150b may be created by welding or soldering the first crown 114b of the first turn 134 to the adjacent crown 114c in the second turn 136. As used herein, “welding” and “soldering” are defined as heating an additional material that is separate from the portions of the stent 120, e.g., the selected crowns 114, the adjacent crowns 114a, the crowns 114b, 114c, or the ends 116, 118, to be welded together and applying the heated additional material to the selected portions of the stent 120, so that when the additional material cools, the selected portions of the stent 120 are welded or soldered together.

In an embodiment, the connections 150, 150a, 150b may be created by fusing, welding, or soldering an additional piece of material (not shown) that extends between selected portions of the stent 120 to be connected. The additional piece of material may resemble a strut or a portion of a strut, and may be sized to provide spacing between the selected crowns of two adjacent turns, if desired. The illustrated embodiments are not intended to be limiting in any way.

The size of the connections 150, 150a, 150b may also be varied according to the desired flexibility and rate of expansion for a given area of the stent 120. In general, the larger the connection, i.e. the larger the fusion or weld, the greater the stiffness, and the slower the rate of expansion of the stent in the area of the larger connections.

It has been found that by providing the additional crown 114b at the end of the wave form 110 and connecting the additional crown 114b to an adjacent crown 114c, as well as connecting the end 116 of the wave form 110 to the crown 114a that lies at the end of the stent 120, as illustrated in FIGS. 3 and 4, a smaller gap will be created when the stent 120 is expanded at the desired deployment site in a vessel, as compared to the configuration illustrated in FIG. 2. The additional connection 150b at each end of the stent 120 may also increase the radiopacity of the ends of the stent so that the ends of the stent 120 may be used as enhanced radiopacity markers for the stent 120.

The embodiments of the stents discussed above may be formed from a wire or a strip of suitable material. In certain embodiments, the stents may be formed, i.e., etched or cut, from a thin tube of suitable material, or from a thin plate of suitable material and rolled into a tube. Suitable materials for the stent include but are not limited to stainless steel, iridium, platinum, gold, tungsten, tantalum, palladium, silver, niobium, zirconium, aluminum, copper, indium, ruthenium, molybdenum, niobium, tin, cobalt, nickel, zinc, iron, gallium, manganese, chromium, titanium, aluminum, vanadium, and carbon, as well as combinations, alloys, and/or laminations thereof. For example, the stent may be formed from a cobalt alloy, such as L605, super elastic alloys such as MP35N®, Nitinol (nickel-titanium shape memory alloy), ABI (palladium-silver alloy), Elgiloy® (cobalt-chromium-nickel alloy), etc. It is also contemplated that the stent may be formed from two or more materials that are laminated together, such as tantalum that is laminated with MP35N®. The stents may also be formed from wires having concentric layers of different materials. Embodiments of the stent may also be formed from hollow tubes, or tubes that have been filled with other materials. The aforementioned materials and laminations are intended to be examples and are not intended to be limiting in any way.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient roadmap for implementing an exemplary embodiment of the invention, it being understood that various changes may be made in the function and arrangement of members described in an exemplary embodiment without departing from the scope of the invention as set forth in the appended claims.

What is claimed is:

1. A stent comprising:
a wave form comprising a plurality of struts and a plurality of crowns, each crown connecting two adjacent struts, the wave form being wrapped around a longitudinal axis to define a plurality of turns;
a first connection that connects an end of the wave form to an adjacent crown in a first turn that defines an end of the stent; and
a second connection that connects a first crown of the wave form to an adjacent crown in a second turn.

2. The stent according to claim 1, wherein the first crown of the wave form and the end of the wave form are separated by a single strut.

3. The stent according to claim 1, wherein an additional crown is located between the crown in the first turn that is connected to the end of the wave form and the crown in the second turn that is connected to the first crown of the wave form.

4. The stent according to claim 1, wherein the plurality of turns are disposed relative to the longitudinal axis at an angle to define a helical coil.

5. The stent according to claim 4, wherein crowns that define the end of the stent lie in a plane substantially perpendicular to the longitudinal axis.

6. The stent according to claim 1, wherein the wave form comprises a single wire.

7. The stent according to claim 1, wherein the first connection is a fusion.

8. The stent according to claim 1, wherein the first connection is a weld.

9. The stent according to claim 1, wherein the second connection is a fusion.

10. The stent according to claim 1, wherein the second connection is a weld.

11. A method of manufacturing a stent, the method comprising:
forming a wave form having a plurality of struts and a plurality of crowns, each crown connecting two adjacent struts;
wrapping the wave form about a longitudinal axis to define a plurality of turns so that an end of the wave form is positioned adjacent a crown of a first turn that defines an end of the stent;
connecting the end of the wave form to the adjacent crown of the first turn; and
connecting a first crown of the wave form to an adjacent crown of a second turn.

12. The method of claim 11, wherein the first crown of the wave form and the end of the wave form are separated by a single strut.

13. The method of claim 11, wherein an additional crown is located between the connected crown of the first turn and the connected crown of the second turn.

14. The method of claim 11, wherein the connecting the end of the wave form to the adjacent crown of the first turn comprises fusing the end of the wave form to the adjacent crown of the first turn.

15. The method of claim 11, wherein the connecting the end of the wave form to the adjacent crown of the first turn comprises welding the end of the wave form to the adjacent crown of the first turn.

16. The method of claim 11, wherein the connecting the first crown of the wave form to the adjacent crown of the second turn comprises fusing the first crown of the wave form to the adjacent crown of the second turn.

17. The method of claim 11, wherein the connecting the first crown of the wave form to the adjacent crown of the second turn comprises welding the first crown of the wave form to the adjacent crown of the second turn.

18. The method of claim 11, wherein the wrapping of the wave form about the longitudinal axis comprises wrapping the wave form at an angle between 0° and 90° relative to the longitudinal axis to create a helical coil.

19. The method of claim 18, wherein the first turn is wrapped about the longitudinal axis so that crowns that define the end of the stent lie in a plane substantially perpendicular to the longitudinal axis.

20. The method of claim 11, wherein the wave form is formed from a single wire.
